(19) United States
(12) Patent Application Publication
(10) Pub. No.: US 2012/0330129 A1
(43) Pub. Date: Dec. 27, 2012

(54) MEDICAL VISUALIZATION SYSTEMS AND RELATED METHODS OF USE

(76) Inventor: Richard AWDEH, Miami Beach, FL (US)

(21) Appl. No.: 13/530,953

(22) Filed: Jun. 22, 2012

Related U.S. Application Data
(60) Provisional application No. 61/500,207, filed on Jun. 23, 2011.

(51) Int. Cl. A61B 1/04; A61B 5/055; A61N 7/00; A61B 6/02; A61B 8/13; A61N 5/067; A61B 6/00; A61B 6/03

(52) U.S. Cl. 600/411; 600/476; 600/427; 600/440; 600/109

(57) ABSTRACT

Embodiments of the disclosure relate to medical devices and, in particular, to medical visualization systems and methods of use. In one embodiment, a medical visualization system may include a video source configured for insertion into a patient and an external data source. A central processing unit in communication with the video source and the external data source may be configured to merge data from the video source and data from the external data source into a left hybrid image and a right hybrid image. The medical visualization system may further include eyewear having left and right oculars in communication with the central processing unit. The left ocular and right ocular may each include a display, and the displays may be configured to project the left hybrid image on the left display and the right hybrid image on the right display.

### CROSS-REFERENCE TO RELATED APPLICATIONS

This application claims the benefits of priority under 35 U.S.C. §§119 and 120 to U.S. Provisional Application No. 61/500,207, filed on Jun. 23, 2011, the entirety of which is incorporated herein by reference.

### FIELD OF THE DISCLOSURE

Embodiments of the present disclosure relate to the field of medical devices and, in particular, to devices for medical visualization and navigation systems. More specifically, embodiments of the present disclosure are directed to software, hardware, and eyewear capable of incorporating external data into a wearer's field of vision in order to create a three-dimensional, surgical-guidance system.

### BACKGROUND OF THE DISCLOSURE

Medical visualization systems, for instance medical microscopes or medical cameras, allow a user to view, e.g., a field, that is in the light path on which the visualization system is focused. During a procedure, however, a surgeon may need to view multiple images simultaneously, for instance, in side-by-side comparison or superimposed, or may need to keep track of information (e.g., data) located outside of the light path or field of view. For instance, a surgeon may require confirmation of anatomical and/or surgical landmarks, e.g., navigational assistance, or may require confirmation of anatomical locations, e.g., to identify cancer margins during diagnosis or tumor resection. Such information may include real-time or static information, such as other images, e.g., magnetic resonance imaging (MRI), computed tomography (CT), optical coherence tomography (OCT), x-ray, or fluorescien angiography (FA) images, patient data, e.g., vital signs or medical records, and/or operation parameters of one or more medical instruments. The ability to incorporate external data into a surgeon's image space in order to relay data points to the surgeon without the surgeon looking away from the surgical field is of great value. Exemplary visualization systems are described, for instance, in U.S. Pat. No. 7,800,820, granted to the inventor hereof, the entirety of which is incorporated by reference herein. There remains a need, however, for visualization systems capable of incorporating external data into the surgeon's field of vision via, for instance, eyewear that a surgeon or other medical professional may wear during medical procedures. Further, there is a need for a surgical navigation system capable of incorporating external data points, such as three-dimensional data points and registered anatomical and pathological landmarks, into a surgeon's field of vision for real-time navigational assistance.

When viewing an image through a microscope or other similar viewing device, an operator can directly view a real-time, live image located within the light path of the device. This image may appear three-dimensional due to the nature of binocular vision, because the glass viewing lenses are situated directly in front of each eye. Such a viewing arrangement may not be possible when the medical camera or other image capture device is located at a distance from the viewer, for instance, within a patient. In this case, external displays set some distance from the image capture device, such as monitors or medical eyewear, may be utilized. The image capture device may relay information to external processors and/or displays for operator viewing. Such displays are two dimensional, and an image is created using pixels on the display. Thus, unlike microscopes or more direct viewing devices, the displayed image may not appear three dimensional. During medical procedures, for instance, an operator may require three-dimensional images to efficiently treat or diagnose a patient. Thus, a need remains for medical eyewear capable of producing three-dimensional images that may be integrated with external data.

### SUMMARY OF THE DISCLOSURE

Embodiments of the present disclosure relate to the field of healthcare medical devices and, in particular, to devices for medical visualization systems.

In one embodiment, a medical visualization system may include a video source configured for insertion into a patient and an external data source. A central processing unit in communication with the video source and the external data source may be configured to merge data from the video source and data from the external data source into a left hybrid image and a right hybrid image. The medical visualization system may further include eyewear having left and right oculars in communication with the central processing unit. The left ocular and right ocular may each include a display, and the displays may be configured to project the left hybrid image on the left display and the right hybrid image on the right display.

Various embodiments of the medical device may include one or more of the following features: the displays may be organic light-emitting displays; the external data source may include one of a magnetic resonance imaging unit, computed tomography unit, optical coherence tomography unit, x-ray machine, ultrasound unit, laser surgical device, or phacoemulsification unit, and the video source may be a camera; and the video source may include two cameras offset from one another along an axis perpendicular to a direction of insertion, wherein images from the cameras are combined to provide a three-dimensional image.

In another embodiment, a medical visualization system may comprise a central processing unit. An imaging source having two cameras may be operably coupled to the central processing unit. An external data source and goggles may also be operably coupled to the central processing unit. The goggles may include one or more displays. The central processing unit may receive data transmitted by the imaging source and the external data source, process the data, and then output a combined data image that is projected onto the displays of the goggles.

Various embodiments of the medical device may include one or more of the following features: the cameras may be mounted on an elongated device and configured for insertion into a patient; the combined data image displayed in the goggles may include data from the external source superimposed on an image from the imaging source; the combined data image may include data from the external data source arranged adjacent to an image from the imaging source; the medical visualization system may include an external monitor configured to receive and display the combined data image from the central processing unit; the central processing unit may be configured to allow an operator to adjust the image displayed in the goggles; the combined data image may include a three-dimensional view of a surgical field; and the imaging source, the external data source, and the goggles may be wirelessly coupled to the central processing unit.

In another embodiment of the present disclosure, a medical visualization and navigation system may include a camera unit configured for insertion into a patient, an external data source, and a central processing unit in communication with the camera unit and the external data source. The central processing unit may be configured to merge data from the camera unit and the external data source with navigational data stored in the central processing unit. The system may also include eyewear having one or more displays in communication with the central processing unit. The central processing unit may create a merged, three-dimensional image and may transmit that image to the eyewear for displaying on the one or more displays.

Various embodiments of the medical device may include one or more of the following features: the central processing unit may compare data from the camera unit with the stored navigational data and identify anatomical landmarks or abnormalities; the merged, three-dimensional image displayed in the eyewear may include data from the external data source and the stored navigational data superimposed on an image from the camera unit; the merged, three-dimensional image displayed in the eyewear may include an indicator to identify the anatomical landmarks or abnormalities; the external data source may include an optical coherence tomography imaging unit configured for insertion in a patient, which in some embodiments may also be configured for use with certain biomarkers; the camera unit, the external data source, and the eyewear may be wirelessly connected to the central processing unit; and the stored navigational data may include anatomical reference images.

Software and hardware processing units may be used to analyze images that may be captured by a video capture source. The software and hardware processing units may be able to register live anatomical data points from these images and compare the data points to anatomy databases in order to create a three-dimensional, registered navigation system for use by a surgeon intraoperatively. The software and hardware processing units may also be capable of incorporating data acquired by imaging devices, e.g., OCT images, that may be combined with contrast agents and/or biomarkers. When used with filters on the eyewear, the eyewear may be able to indicate anatomical margins and/or tumor margins to a wearer, for example, in the case of solid tumors.

Moreover, those skilled in the art will appreciate that the conception upon which this disclosure is based may readily be used as a basis for designing other structures, methods, and systems for carrying out the several purposes of the present disclosure. It is important, therefore, to recognize that the claims should be regarded as including such equivalent constructions insofar as they do not depart from the spirit and scope of the present disclosure.

### BRIEF DESCRIPTION OF THE DRAWINGS

The accompanying drawing illustrates certain exemplary embodiments of the present disclosure, and together with the description, serves to explain principles of the present disclosure.

FIG. 1 depicts a schematic view of an exemplary visualization system, in accordance with an embodiment of the present disclosure.

### DETAILED DESCRIPTION OF THE PREFERRED EMBODIMENTS

Reference will now be made in detail to the exemplary embodiments of the present disclosure described below and illustrated in the accompanying drawing. Wherever possible, the same reference numbers will be used throughout to refer to same or like parts.

While the present invention is described herein with reference to illustrative embodiments for particular applications, it should be understood that the invention is not limited thereto. Those having ordinary skill in the art and access to the teachings provided herein will recognize additional modifications, applications, embodiments, and substitution of equivalents, that all fall within the scope of the disclosure. Accordingly, the disclosure is not to be considered as limited by the foregoing or following descriptions.

Other features and advantages and potential uses of the present disclosure will become apparent to someone skilled in the art from the following description of the disclosure, which refers to the accompanying drawings.

Prior to providing a detailed description of the embodiments disclosed herein, however, the following overview is provided to generally describe the contemplated embodiments. Principles of the present disclosure may be suitable for use in a range of applications, including, e.g., for use with endoscopes or any suitable introduction sheath with visualization capabilities. Further, although the embodiments disclosed herein are described in connection with medical visualization systems, those of ordinary skill in the art will understand that the principles of the present disclosure may be suitable for nonmedical applications, such as, e.g., the inspection or repair of machinery and military operations. In addition, the principles of the present disclosure may be suitable for the display of data, graphics, and video, both dynamic and static, inside any type of visualization system, for instance, nonsurgical microscopes or cameras, or systems including those for, e.g., video games, virtual reality devices, vision-enhancing goggles—e.g., goggles using infrared or ambient light to aid vision—telescopes, binoculars, and so forth.

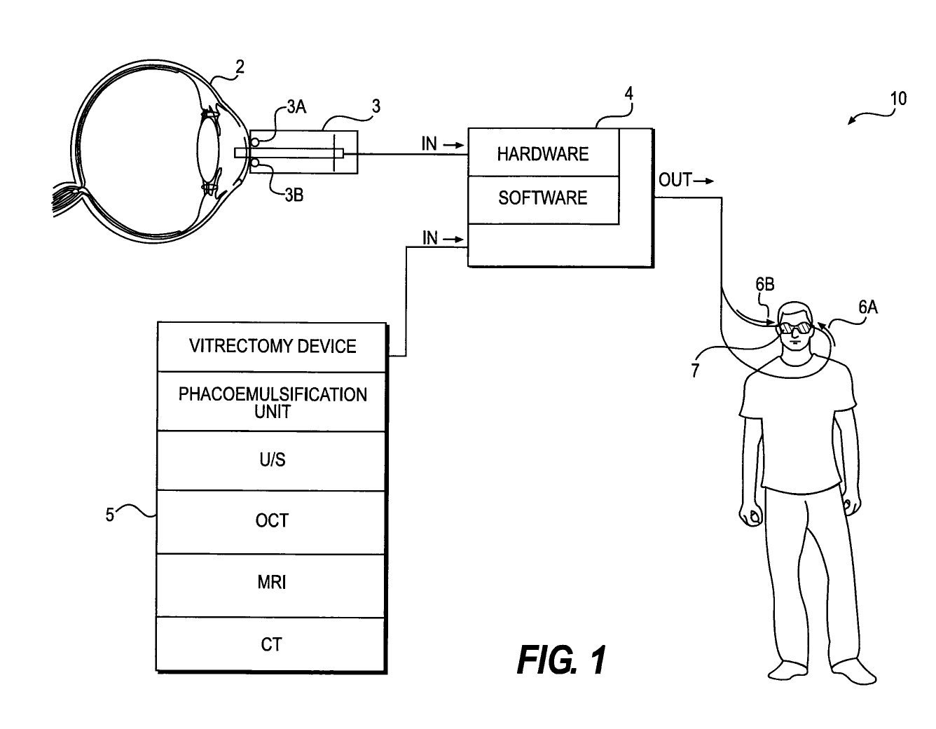

FIG. 1 illustrates a medical visualization system 10, according to an exemplary embodiment. Medical visualization system 10 may include a video capture source 3 comprised of one or more medical cameras. Video capture source 3 may generate still images, moving images, or both. In one embodiment, for instance, video capture source 3 may include two cameras, 3a, 3b. The cameras may be slightly offset and coupled together to provide a three-dimensional view of the surgical field. Medical cameras 3a, 3b may include any suitable type of camera, e.g., cameras for generating still or moving images, infrared or heat-sensitive cameras, low-light cameras, or the like. Medical visualization system 10 may also include any suitable component for visualization and/or imaging, e.g., one or more light sources, sensors, or suction/irrigation sources to clear the visual field, for instance.

Video capture source 3 may be configured for insertion into the human body, such as, for example, into an eye 2 (e.g., through the sclera and into the anterior chamber or vitreous space), the abdomen (e.g., through the abdominal wall and into the abdominal cavity), or into any suitable body lumen or body cavity, e.g., the gastrointestinal or esophageal tracts and the oral, anal, or vaginal cavities, so as to allow an operator to view the internal anatomies of a patient. In some embodiments, cameras 3a, 3b may be coupled to or embedded in an elongated, shaft-like device to aid insertion into the body. Video capture source 3 may be configured for introduction into the body through, for instance, a trocar, a catheter, a guide tube, an endoscope, or any suitable introduction means. Video capture source 3 may be configured to access the body through a natural orifice or through an incision made in the body during either laparoscopic, endoscopic, or traditional invasive procedures, for example. In particular, certain embodiments of the present disclosure may be used in, or prior to, ophthalmologic surgeries, including vitreo-retinal surgery (e.g., with phacoemulsification, ultrasound, vacuum, aspiration, or irrigation), corrective surgery (e.g., Laser-Assisted in Situ Keratomileusis, or LASIK, and photorefractive keratectomy, or PRK), cataract surgery, glaucoma surgery, or in any other suitable procedures in any other subspecialties, for instance, other surgical fields or dentistry.

Medical visualization system 10 may further include eyewear 7. Eyewear 7 may include eyeglasses, spectacles, goggles, a helmet, visors, monocles, or any other suitable wearable viewing device. Eyewear 7 may be operably connected to video capture source 3 so that images from the cameras are displayed in eyewear 7 to provide visualization of a surgical field, for instance, to the wearer. Eyewear 7 may be physically connected (e.g., via cords, cables, wires, or the like) or wirelessly connected to video capture source 3 via a central processing unit 4, described further below. Eyewear 7 may include one or more displays for displaying the images from video capture source 3. The displays may be, e.g., liquid crystal displays (LCDs) or light-emitting diode (LED) displays, and may include, for instance, one or more of an organic light-emitting diode (OLED), a transparent organic light-emitting diode (TOLED), or any other suitable light source. Eyewear 7 may, for instance, include any suitable OLED display and/or control systems, such as those marketed by eMagin Corporation, 3006 Northup Way, Suite 103, Bellevue, Wash. 98004.

The one or more displays may be located in each of the eyewear oculars. In one embodiment, each display may have its own video stream 6a, 6b, which may allow for the delivery of data or an image (still, video, or both) signal to each ocular, as discussed further below. Alternatively, each display may share one or more video feeds 6a, 6b. In another embodiment, eyewear 7 may only have one display and/or one ocular. The oculars can be transparent, semi-transparent, translucent, or semi-translucent, so that the display image is included in what the user can see through the oculars; or, the oculars can be opaque or semi-opaque, such that the display image is the only image the user can see. In some embodiments, eyewear 7 may include controls configured to allow a wearer to adjust the image displayed on one or both oculars. For instance, a user may be able to zoom in or out of an image, adjust the brightness, contrast, color, or magnification, or completely turn off the display in one or both oculars. This may allow a wearer to view the image displayed on, e.g., one ocular, while keeping an eye on something else, for instance, when reaching for an instrument or viewing another region of the patient.

Medical visualization system 10 may be configured to incorporate both imaging data from video capture source 3 and external data, from, for instance, an external data source 5, into the visual field of an operator wearing eyewear 7. Any external data that may assist a medical professional during a medical procedure may be included on the displays within eyewear 7. Such external data may include real-time or static information, such as other images, e.g., magnetic resonance imaging (MRI), computed tomography (CT), optical coherence tomography (OTC), x-ray, or fluorescein angiography (FA) images; patient data, e.g., physiological parameters or medical records; and/or parameters of or information particular to one or more medical instruments being used, e.g., phacoemulsification machines for cataract surgery, surgical lasers for eye surgery, specifically femtosecond and excimer laser devices, or thermocautery devices.

Central software/hardware processing unit 4 may acquire data wirelessly or via a physical connection from one or more video capture sources 3 and/or from one or more external data sources 5 (MRI, CT, ophthalmology/phacoemulsification data stream, medical instruments, patient monitors, cameras, etc.) and may incorporate this data onto the video source data, forming a merged image, for instance, by superimposing the data or arranging the data into discrete images adjacent to each other. In addition to the view of the, e.g., surgical field, provided by video capture source 3, one or more data images may be produced in the view provided to the user's eyewear, thus permitting the user to simultaneously view both the object(s) on which the camera(s) is trained, as well as external data. The external data can include, for instance, vacuum pressure, distance to an object or anatomical landmark, or other suitable information. Still other information could include, for example, the remaining battery life of eyewear 7 or the time or length of the current operation.

The one or more displays in eyewear 7 may be capable of receiving either merged or unmerged data from central processing unit 4. For instance, live video images from cameras 3a, 3b may be displayed in each ocular of eyewear 7 or images from 3a, 3b may be merged in central processing unit 4 to form one, merged image for display on one or more oculars. In one embodiment, cameras 3a, 3b may be offset by a pre-specified number of degrees to create a three-dimensional image in eyewear 7. The images acquired from video capture source 3 may further include data, such as images or values, from external data source 5 (e.g., a medical device such as an MRI scan, ultrasound machine, vacuum, etc.).

As alluded to above, each set of images or information from external data source 5, video capture source 3, and/or visualization system 10 may be processed, compiled, and repositioned in central processing unit 4. The combined image may then be delivered to the oculars on eyewear 7. For instance, the image received from video capture source 3 may be surrounded by a dark, black, or transparent area. Thus, the image of the surgical field from video capture source 3 may include blank, non-image portions. In one embodiment, external data from external data source 5 may be added to the visual field in this blank space on the images from video capture source 3. In other embodiments, central processing unit 4 may remove these blank portions and may superimpose the external data onto the view of the surgical field from video capture source 3. External data may be incorporated on the edge of a display in eyewear 7 such that the central portion of the video image delivered to the operator includes a surgical field, for instance, and the periphery includes the external data. In one embodiment, the external data may be delivered to just one ocular, while the other ocular may receive a continuous feed from video capture source 3. In another embodiment, data from external data source 5 may be delivered to both oculars and may be superimposed over the image from video capture source 3 in both oculars. This embodiment may be used, for instance, to superimpose a flourescien angiogram of blood vessels over the real-time image of blood vessels during retina surgery or any other suitable procedure. Additional exemplary configurations of the images displayed by each ocular of the eyewear are described in reference to the microscope system in U.S. Pat. No. 7,800,820, for example.

Central processing unit 4 may merge data received from both video capture source 3 and external data source 5 into a left hybrid image and a right hybrid image. The composite display image, including the processed and repositioned images from video capture source 3 and any other information, may then be sent to the displays in eyewear 7. In one embodiment, external data source 5 may include a sensor located on one or more medical instruments configured to transmit orientation information to central processing unit 4. Such orientation information may aid central processing unit 4 to align and/or orient the images received from video capture source 3. Alternatively, the orientation information could simply be displayed in eyewear 7 to indicate the orientation of the field of view to the wearer of eyewear 7.

In one embodiment, eyewear 7 may include left and right oculars in communication with central processing unit 4, and each ocular may have its own display. Eyewear 7 may be configured to project the left hybrid image on the left display and the right hybrid image on the right display. For instance, two data streams 6a, 6b may operably connect central processing unit 4 and eyewear 7. A left data stream 6a may project the left hybrid image from central processing unit 4 on the left display of eyewear 7, and the right data stream 6b may project the right hybrid image from central processing unit 4 on the right display of eyewear 7. In another embodiment, external or video source data and/or images may be displayed in one ocular, while the other ocular may allow a user to see through the display. While two video streams 6a, 6b are shown in the exemplary FIGURE, any number of video streams may be used. The images may be sent using any suitable video format, such as, for example, digital, 12-bit video, NTSC, PAL, SECAM, or stereoscopic video. Central processing unit 4 may further include memory for storing external data, images from video capture source 3, and/or the final composite images sent to eyewear 7. Central processing unit 4 may allow an operator to record these images, pause the images, re-orient the images, or otherwise control the images displayed either in real-time or after the images have been recorded. Further, the raw data from video capture source 3 and external data source 5, and the composite images may be transmitted to an external processor and/or display monitor, located either in the same room or in a remote area. For instance, images may be transmitted to an external monitor to allow people other than the wearer of eyewear 7 to view the images.

While FIG. 1 depicts central processing unit 4 as separate from eyewear 7, central processing unit 4 may also be included in eyewear 7. In this embodiment, data from video capture source 3 and/or external data source 5 may stream directly into eyewear 7, and all remaining processing may be performed in eyewear 7. In other embodiments, some data processing may occur in a central processing unit 4 located external from eyewear 7, while some processing may occur within a central processing unit 4 located within eyewear 7.

In certain embodiments, a wearer may be able to adjust controls on eyewear 7 to view only data from video capture source 3 or to view only data from external data source 5 in eyewear 7, alternatively, or to view external data in one ocular and video capture data in the other, or to stop the display of data from all sources. In one embodiment, the physical organization of eyewear 7 may allow a user to adjust the data displayed. For instance, eyewear 7 may include an outer display portion and an inner display portion. Eyewear 7 may be configured such that external data is displayed on one of the inner or the outer display portions, and images from video capture source 3 are displayed on the other of the inner or outer display portion. For instance, external data may be displayed on the outer portion, and images from video capture source 3 may be displayed on the internal portion. The outer portion may be configured so that a wearer may move the outer portion in relation to the inner portion, altering the orientation of the displayed external data relative to the displayed images from video capture source 3. For instance, in one embodiment, an outer display portion may be, e.g., slidingly, pivotably, or hingedly coupled to the inner portion such that a wearer may view both the external data and data from video capture source 3, or alternatively, position the outer portion of eyewear 7 such that only one of either the internal or external data can be viewed by a wearer. In another embodiment, both the outer portion and the inner portion may be movable, or the external data and internal data from video capture source 3 may both be displayed on the outer portion. In this embodiment, the wearer may be able to move the displayed images into and out of the wearer's visual field. This may allow an operator to alternatively navigate the surrounding environment of the operating room and view the surgical area and/or external data without having to completely remove eyewear 7.

Additionally, eyewear 7 may be sized and/or shaped so as to allow a wearer to quickly glance outside of the ocular and/or display region, for instance, to glance just below the display, and view the immediate field through eyewear 7. Alternatively, in one embodiment, eyewear 7 may have multiple oculars and/or displays arranged in rows or columns that are configured to display different external data or data from different external data sources 5 to allow a wearer to glance between each. The displays of this embodiment may be arranged in a manner similar to bifocals, for instance.

Eyewear 7 may eliminate the need for a microscope in an operating room. Microscopes are necessary for many fine procedures, including eye surgeries, and more specifically, for those procedures involving lasers. The ability to use eyewear 7 instead of a microscope for such procedures may decrease the cost of maintaining medical facilities, which may allow operators to perform these procedures in non-hospital environments, such as clinics. Further, eyewear 7 may provide a light-weight, portable alternative to traditional medical visualization systems. This may allow an operator greater freedom of movement. For instance, eyewear 7 may allow on operator to be in a remote location, for instance, in another room, in the case of robotic surgery. Additionally, numerous eyewear 7 may be connected to central processing unit 4, which may allow multiple operators and/or multiple observers in any location to view the combined images.

Further, eyewear 7 may enhance a wearer's ability to perform procedures. In one embodiment, eyewear 7 may include a filter, or other lighting optimization component, that makes it easier for a wearer to view certain structures while operating. For instance, a blue filter may allow an operator to more easily view sutures, for instance, e.g., 10-0 nylon sutures. In another embodiment, eyewear 7 or central processing unit 4 may include processing that allows for real-time ‘matching’ or recognition of anatomical landmarks for navigational assistance. For instance, images from video source 3 may be compared with external data, e.g., with the pre-operative MRI/data studies of anatomical landmarks in the field, and the display shown on eyewear 7 may ‘recognize’ and indicate these landmarks for an operator. In another embodiment, central processing unit 4 may include anatomical maps or anatomical reference images. Algorithms may allow central processing unit 4 to compare data from video capture source 3 with these anatomical maps, and the display shown on eyewear 7 may indicate certain landmarks or abnormalities in the surgical field to a wearer, for instance, through visual or auditory signals.

In another embodiment, eyewear 7 may be used in procedures utilizing a contrast agent that is bound to an antibody, or any suitable biomarker, e.g., in procedures involving cancer. In this embodiment, eyewear 7 may include filters configured for use with contrast agents, or may have multiplexing abilities to allow for the use of multiple contrast agents. The filters, e.g., may correspond to the biomarkers used such that eyewear 7 may allow a wearer to distinguish tumor sections bound by the biomarkers from unbound, non-tumor areas (i.e., to see the ‘tumor margins’) on the image display to permit a wearer to perform more exact diagnoses or surgical excisions, for instance. Exemplary use of such biomarkers is described, for example, in PCT Patent Publication No. WO 2010/148298, of which the inventor hereof is a co-inventor, and the entirety of which is incorporated by reference herein. As disclosed in the PCT application, contrast agents may be used with optical coherence tomography (OCT) imaging to map anatomies or abnormalities, e.g., of the eye. Accordingly, in one embodiment, an OCT probe, e.g. a fiber optic cable, may be configured for insertion into a patient with video capture source 3. The OCT probe may be mounted, for example, on a catheter tip or inserted through an introduction sheath, such as an endoscope or trocar. Alternatively, the OCT probe may be mounted on the elongated device on which cameras 3a, 3b may be mounted. The OCT probe may act as an external data source and may transmit images to central processing unit 4 and eyewear 7. Central processing unit 4 may merge the images from video capture source 3 and the OCT probe so that the OCT images are superimposed on the surgical field as displayed in eyewear 7. A three-dimensional image with OCT mapping may be generated and streamed to the displays in eyewear 7. This may offer a wearer OCT image guidance, which may further be multiplexed to contrast agents that are bound to areas within a patient.

Systems and methods embodying principles of the present disclosure can allow the operator the benefit of having data superimposition in real-time over the visual surgical field. Additionally, it may give the operator the ability to perform camera-based operations in a three-dimensional plane, as compared to current two-dimensional technologies.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents that all fall within the scope of the embodiments described herein. Accordingly, the embodiments described herein are not to be considered as limited by the foregoing description.

What is claimed is:

1. A medical visualization system comprising:
a video source configured for use with a patient;
an external data source;
a central processing unit in communication with the video source and the external data source, the central processing unit being configured to merge data from the video source and data from the external data source into a left hybrid image and a right hybrid image; and
eyewear including left and right oculars in communication with the central processing unit, the left ocular and the right ocular each including a display, the displays being configured to project the left hybrid image on the left display and the right hybrid image on the right display.

2. The medical visualization system of claim 1, wherein the displays include one of either light-emitting diode displays, organic light-emitting diode displays, transparent organic light-emitting diode displays, or liquid crystal displays.

3. The medical visualization system of claim 1, wherein the external data source is one of a magnetic resonance imaging unit, computed tomography unit, optical coherence tomography unit, x-ray machine, ultrasound unit, a surgical laser device, or phacoemulsification unit, and the video source is a camera configured for insertion in a patient.

4. The medical visualization system of claim 1, wherein the video source includes two cameras offset from one another along an axis perpendicular to a direction of insertion, wherein images from the cameras are combined to provide a three-dimensional image.

5. A medical visualization system comprising:
a central processing unit;
an imaging source operably coupled to the central processing unit, wherein the imaging source includes two cameras;
an external data source operably coupled to the central processing unit; and
goggles operably coupled to the central processing unit, wherein the goggles include one or more displays, and
wherein the central processing unit receives data transmitted by the imaging source and the external data source, the central processing unit processes the data, and the central processing unit outputs a combined data image that is projected onto the displays of the goggles.

6. The medical visualization system of claim 5, wherein the cameras are mounted on an elongated device and configured for insertion into a patient.

7. The medical visualization system of claim 5, wherein the combined data image displayed in the goggles includes the data from the external data source superimposed on an image from the imaging source.

8. The medical visualization system of claim 5, wherein the combined data image displayed in the goggles includes the data from the external data source arranged adjacent to an image from the imaging source.

9. The medical visualization system of claim 5, further including an external monitor operably coupled to the central processing unit and configured to receive and display the combined data image.

10. The medical visualization system of claim 5, wherein the central processing unit is configured to allow an operator to adjust the image displayed in the goggles.

11. The medical visualization system of claim 5, wherein the combined data image includes a three-dimensional view of a surgical field.

12. The medical visualization system of claim 5, wherein each of the imaging source, the external data source, and the goggles are wirelessly coupled to the central processing unit.

13. A medical visualization and navigation system comprising:
a camera unit configured for insertion into a patient;
an external data source;
a central processing unit in communication with the camera unit and the external data source, the central processing unit being configured to merge data from the camera unit, data from the external data source, and navigational data stored in the central processing unit; and
eyewear having one or more displays in communication with the central processing unit,
wherein the central processing unit creates a merged, three-dimensional image and transmits this image to the eyewear for displaying on the one or more displays.

14. The medical visualization and navigation system of claim 13, wherein the central processing unit compares the data from the camera unit with the navigational data stored in the central processing unit and identifies anatomical landmarks or abnormalities.

15. The medical visualization and navigation system of claim 13, wherein the merged, three-dimensional image displayed in the eyewear includes the data from the external data source and the navigational data stored in the central processing unit superimposed on an image from the camera unit.

16. The medical visualization and navigation system of claim 13, wherein the merged, three-dimensional image displayed in the eyewear includes an indicator to identify the anatomical landmarks or abnormalities.

17. The medical visualization and navigation system of claim 13, wherein the external data source is an optical coherence tomography imaging unit configured for insertion into a patient.

18. The medical visualization and navigation system of claim 17, wherein the optical coherence tomography imaging unit is configured for use with certain biomarkers.

19. The medical visualization and navigation system of claim 13, wherein the camera unit, the external data source, and the eyewear are wirelessly connected to the central processing unit.

20. The medical visualization and navigation system of claim 13, wherein the navigational data stored in the central processing unit includes anatomical reference images.
